(19) United States
(12) Patent Application Publication
(10) Pub. No.: US 2006/0287257 A1
(43) Pub. Date: Dec. 21, 2006

(54) Pharmaceutical compositions to treat diseases caused by mycobacterium

(76) Inventor: Richard F. Stockel, Bridgewater, NJ (US)

Correspondence Address: RICHARD F. STOCKEL, 475 ROLLING HILLS ROAD, BRIDGEWATER, NJ, 08807, US

(21) Appl. No.: 11/454,082

(22) Filed: Jun. 15, 2006

Related U.S. Application Data
(60) Provisional application No. 60/693,600, filed on Jun. 20, 2005.

(51) Int. Cl. A61K 31/7048; A61K 31/7034; A61K 31/675; A61K 31/663; A61K 31/43; A61K 31/496

(52) U.S. Cl. 514/27; 514/28; 514/35; 514/79; 514/80; 514/114; 514/291; 514/253.08; 514/200; 514/192; 514/356; 514/312; 514/300

(57) ABSTRACT

The invention teaches the synthesis of pharmaceutical composition and methods of synthesis to treat people and animals infected with a pathogenic mycobacterium. In particular the compositions of this invention are suitable for the treatment of tuberculosis, malaria, and other infections diseases caused by mycobacterium.

### BACKGROUND OF THE INVENTION

1. Field of the Invention

This invention relates to the synthesis and methods to synthesize pharmaceutical drugs to treat various diseases caused by the mycobacterium such as tuberculosis, malaria and other infectious diseases.

2. Background of the Prior-Art

Tuberculosis is caused by infection with mycobacterium tuberculosis, and malaria by plasmodium falciparum.

It is estimated that about one-third of new tuberculosis cases are resistant to current drug treatment regimens, and estimates are that drug-resistant tuberculosis accounts for between 2% and 14% of total tuberculosis cases worldwide.

Similarly drug-resistant in treating malaria is also occurring at a rapid rate. It estimated that about one million people die each year due to disease. Chloroquine has encounter difficulties in properly and effectively treating malaria.

Recently, drug combinations for multi-drug resistant malaria are being developed, e.g., atovaquone plus proguanil and artemether plus benfhimetol.

In addition to tuberculosis and malaria, there are a number of other human and animal diseases caused by mycobacteria, including leprosy, lymphadenitis, a variety of pulmonary and skin diseases and would infection. Resistance to drugs used in current practice has now produced an immediate need for more effective drugs against many different mycobacterium species.

### SUMMARY OF THE INVENTION

The present invention is directed to improved anti-mycobacterium compositions and methods for their preparation. In particular, the invention is directed towards the synthesis of new anti-mycobacterial drugs by forming complexes of bioactive acid salts of amino containing drugs with bioactive phenolates, carboxylates, dialkyldithiocarbamates, mercaptides, organic phosphates, organic phosphonates, organic phosphinates, organic sulfonates and other bioactive anions which can cause a precipitate when it combines with the bioactive amines cation in the appropriate solvent system. The synthesis of the complexes of this invention are usually carried out in aqueous or alcoholic-aqueous medium by reacting a water-soluble or partially water-soluble acid salt of an anti-mycobacterial amino molecule with the sodium salt of a halogenated phenolic molecule. This type of reaction is known as metathesis. The novelty of this approach is that the complex now has an active drug as the cation and a second drug as the anion.

Another type of reaction, which allows some of the complexes to be synthesized is an acid-base reaction. If the reactants can undergo a protonation transfer, then a simple acid-base reaction can be utilized in preparing some of the bioactive complexes of this invention as well.

### ADVANTAGE OF THE INVENTION

There are several advantages in treating diseases with the complexes taught in this invention. They are:
Complexes have multiple target sites in the organism to attack, thereby lessening the chance of the microbe or parasite surviving the therapeutic treatment,
Complexes are neutral molecules therefore they can penetrate the cell wall more easily than cationic drugs,
The hydrophilic-lipophilic balance of the complex can be controlled, thereby altering they permeability of the drug to penetrate skin, mucosa, etc.,
Inexpensive, since the vast majority of the cationic and anionic molecules are commercially available and produced in bulk
The vast majority of the cationic and anionic molecules are FDA, and/or EPA approved, whereby lessening toxicity concerns

The pharmakinetics (rate of delivery) will depend on the solubility constant (pKsp) of said complex. Depending on this pKsp, the treatment can be controlled over a specified time period. In addition to solubility, permeability is equally important, as the drug passes through the GI tract and eventually enters the bloodstream drugs with basic and acidic ionic character will be present in very different ionic forms in different parts of the body. Whereas a degree of ionization is beneficial in improving solubility, the pH-partition theory of permeability suggests that only the neutral form of a compound is available for passive transport across membranes. Thus the drug complexes of this invention being basically neutral should have excellent permeability to their intended targets.

### DETAILED DESCRIPTION OF THE PREFERRED EMBODIMENTS

This invention teaches the formation of combination drug complexes prepared by the metathesis reaction of a bioactive amino salt having at least partial water solubility reacting with a bioactive halophenolate to form the complex and a salt by-product.

Many tuberculosis, malaria and other anti-mycobacterial drugs have one or more basic nitrogen atoms (lone pair of electrons), which can be converted to amino salts. These salts have effective water solubility in order to react with the halophenolate sodium salt or carboxylate sodium salt in aqueous medium or a water-alcohol medium.

Some examples of these amino containing anti-mycobacterial drugs include quaternaries, quinine, chloroquine, primaquine, pyrimethamine, mefloquine, halofantrine, sulfadoxine, dapsone, ciprofloxacin, pefloxacin, norfloxacin, nalidixic acid, plaquenil, isoniazid, ethionamide, pyrazinamide, ethambutal, pentamidino, proquanil, amodiaquin, sulfadoxine, p-aminosalicylic acid, iodoquinol, paromomycin, metronidazole, tinidazole, amphotericin, albendazole, mebendazole, pyrantel, clindamycin, azithromycin, thiabendazole, quinacrine, furazolidone, rifampin and the like. Several polymeric cationic materials can also be utilized in the teachings of this invention. They involved polyguanidines, polybiguanides and polyionenes. Specific compositions, given as examples, include polyhexamethylene guanide, polyhexamethylenebiguanide, Busan-77 and poly (N,N-dimethylhexamethylene) salts, the latter being polyionenes.

These drugs are merely illustrative in scope realizing that many other bioactive molecules can be useful in carrying out the teachings of this invention. The literature is replete with many other reported active compositions.

Some examples, not all inclusive, are U.S. Pat. No. 3,992,446 (guanidine), U.S. Pat. No. 4,031,220 (quinoline), U.S. Pat. No. 4,195,089 (pyridinol), U.S. Pat. No. 5,206,236 (amidine and imidazoline), U.S. Pat. No. 5,817,686 (bis-benzimidazoles), U.S. Pat. No. 6,693,217B2 (N,N′-substituted biguanides derived from hydroxylamines) to illustrate the compositions which are useful to prepare the cationic anti-mycobacterial portion of the drugs of this invention. Pyrrole anti-mycobacterial compounds are additional amine containing compound useful for this invention as reported in “Bioorganic and Medicinal Chemistry 12 (2004) p 1453-1458”. The anionic portion of the anti-mycobacterial drugs of this invention are bioactive molecules, which are capable of reacting with the bioactive amine salt via of metathesis reaction in an appropriate solvent(s) medium. In order for this metathesis reaction to operate, it is essential that one of the products either precipitates or evolves as a gaseous by-product.

Other cationic or anionic anti-mycobacterial drugs include:
1,2,5-oxadiazole or 1,2/1,5-isoxazole molecules as reported in J. Biol. Chem. Y279, No. 30, Is. July 23, p 31429
Tamulin first generation of a pleuromutilin was reported in C & EN Mar. 28, 2005, page 10,
Diarylquinoline (specifically R207910) which inhibits ATP synthase reported in C & EN Dec. 13, 2004, V82, No. 50, page 7
Dicationic amidines synthesized by David W. Boykin and Richard R. Tidwell are active anti-mycobacterial agents,
Benflumetal an anti-malarial drug reported in Am. J. Trop. Med. Hyg. 61 (31, 1999, page 439, and
Revlimid and/or Actimid, which have excellent activity against the mycobacteria causing leprosy.

Suitable reactive bioactive anionic moieties are phenolates, mercaptides, carboxylates, sulfonates, phosphates, phosphonates, phosphinates, 2-hydroxyl-1,4-naphthoquinones, bisphosphonates and the like. Anionic species of the above compositions can be readily formed by treating them with a variety of bases, e.g., alkali hydroxides, alkali carbonate or bicarbonate depending on the acidity of the hydrogen atom being replaced.

In certain cases the complexes of this invention can be prepared by reacting an active amine drug with an active drug capable of donating a proton, e.g., carboxylic acids, alkyl or aryl sulfonic acids, or alkyl or aryl phosphoric, phosphorous, phosphonic, bisphosphonic or phosphinic acids. These examples represent a acid-base reaction, which can be readily prepared by refluxing in an inert solvent, e.g., water, alcohols, acetone, ketone, ethers, esters and aprotic dipolar solvents.

Some specific examples of these bioactive compounds, which can be converted to anions to react with the cationic amine salt, or compounds which can readily donate a proton to the amine substrate are triclosan, o-phenylphenol, thymol, 2-mercapto pyridine n-oxide, dialkyldithio carbamates, lauryl sulfonates, arachidonic acid, docosahexaenoic acid, docosanoic acid, di-2-ethylhexyl phosphoric acid and the like.

A preferred phenolic compound is triclosan. In the past few years several investigators have shown that triclosan is an effective drug for chemotherapy of anti-mycobacertial diseases, specifically against malaria. “Molecular and Cellular Biochemistry 253:55-63,2003”.

Another class of potential anionic bioactive mycobacterial compounds are those with a imide or sulfonamide functionality, which can form an anion by the reaction of a strong base for e.g., solid potassium hydroxide, lithium hydride and the like in a inert solvent (nonhydroxlytic) e.g., ether, THF, glyme and the like. The specific example of a mycobacterial drug molecule having a imide moiety is thalidomide or it derivatives like Revlimid or Actimid to mention a few.

Another anionic functionality, which has indicated good activity with bioactive cations are carboxylates. For example the chain length should be from C₈ to C₂₂, either staturated or unsaturated, and optionally functionalized with an amino, hydroxy, epoxy or halide groups.

A major component of this invention is the ability to form medicinals to kill or inhibit mycobaceteria with compositions containing at least two or more known bioactive molecules combined together via metathesis or acid-base synthesis resulting in an effective treatment by having the capability of destroying these pathogens by more than one mechanism, if need be. This approach will lessen drug resistance and lessen the direction of the drug therapy.

### EXPERIMENTAL

Metathesis Route—in order for this type reaction to achieve satisfactory conversions the product must precipitate from the reaction medium. Usually this can occur in aqueous or aqueous-alcohol solvents. In some cases, other non-hydroxylic solvents are preferred depending on the insolubility of the complex being formed and/or the hydrolysis sensitivity of the alkali salt. Normally metathesis reactions do not require heating (reflux), but this is optional.

Ex: Isoniazide Hydrochloride—Triclosan Complex

0.05 moles of triclosan sodium salt dissolved in 200 ml of water was added to 0.025 moles of isoniazide hydrochloride dissolved in 200 ml of water. A precipitate was formed immediately with a near quantitative yield. The crude product was very pure, and it could be recrystallized from isopropanol-water mixtures. FTIR and nitrogen analysis confirmed it's structure.

Acid-Base Reaction—in order for this reactive to be successful, the bioactive amine molecular must be able to accept a proton from a bioactive molecule having sufficient acidity.

Ex: 0.05 m of proguanil hydrochloride and 0.05 m of arachidonic acid were refluxed in 250 ml of isopropanol for 12-20 hours. Upon evaporating off the solvent white crystals precipitated. Yields were about 90%. FTIR and nitrogen analysis confirmed it's structure.

Tuberculosis Antimicrobial Results

Metathesis Route—in order for this type reaction to achieve satisfactory conversions the product must precipitate from the reaction medium. Usually this can occur in aqueous or aqueous-alcohol solvents. In some cases, other non-hydroxylic solvents are preferred depending on the insolubility of the complex being formed and/or the hydrolysis sensitivity of the alkali salt. Normally metathesis reactions do not require heating (reflux), but this is optional.

Ex: Isoniazide Hydrochloride—Triclosan Complex

0.05 moles of triclosan sodium salt dissolved in 200 ml of water was added to 0.025 moles of isoniazide hydrochloride dissolved in 200 ml of water. A precipitate was formed immediately with a near quantitative yield. The crude product was very pure, and it could be recrystallized from isopropanol-water mixtures. FTIR and nitrogen analysis confirmed it's structure.

Acid-Base Reaction—in order for this reactive to be successful, the bioactive amine molecular must be able to accept a proton from a bioactive molecule having sufficient acidity.

Ex: 0.05 m of proguanil hydrochloride and 0.05 m of arachidonic acid were refluxed in 250 ml of isopropanol for 12-20 hours. Upon evaporating off the solvent white crystals precipitated. Yields were about 90%. FTIR and nitrogen analysis confirmed it's structure.

Tuberculosis Antimicrobial Results

All of the compounds were screened against Mycobacterium tuberculosis strain H₃₇Rv by serial dilution. The minimum inhibitory concentration and degree of inhibition is presented.

| | | MIC | % |
|---|---|---|---|
| Compound | | (ug/ml) | Inhibition |
| 1. | chlorhexidine distearate | 3.13 | 99 |
| 2. | Isoniazide dodecyl benzene sulfonate | 0.78 | 99 |
| 3. | N-cocoylamide-L-argimine ethyl ester | 1.56 | 94 |
| | triclosanate | | |
| 4. | chlorhexidine dithymol | 1.56 | 98 |
| 5. | chlorhexidine di-2-mercaptobenzthiazole | 1.56 | 90 |
| 6. | chlorhexidine dilaurate | 3.13 | 99 |
| 7. | chlorhexidine di [4-amino-1- | 3.13 | 94 |
| | hydroxybutylidene] bis-phosphonate | | |
| 8. | chlorhexidine beta cyclodextrin sulfobutyl ether | 3.13 | 94 |
| 9. | chlorhexidine di-ortho phenyl phenol | 3.13 | 99 |
| 10. | poly (hexamethylene) biguanide stearate | 6.25 | 94 |
| 11. | chlorhexidine distearate | 3.13 | 99 |

I claim:

1. Anti-mycobacterial compositions prepared by a metathesis reaction with a known: anti-mycobacterial drug containing at least one amine salt with a known anti-mycobacterial drug in it's anion form in a solvent system whereby said composition precipitates from said solution.

2. Anti-mycobacterial compositions prepared by an acid-base reaction whereby a known anti-mycobacterial drug containing at least one amino group which is capable in accepting a proton from a anti-mycobacterial acidic drug molecule formed by refluxing said acid and base in a inert solvent.

3. Anti-mycobacterial amine acid salts of claim 1 consisting of quinine, chloroquine, primaquine, pyrimethamine, mefloquine, halofantrine, sulfadoxine, dapsone, ciprofloxacin, pefloxacin, norfloxacin, nalidixic acid, plaquenil, isoniazid, ethionamide, pyrazinamide, ethambutal, pentamidine, proquanil, amodiaquin, sulfadoxine, p-aminosalicylic acid, iodoquinol, paromomycin, metronidazole, timidazole, amphotericin, albendazole, mebendazole, pyrantel, clindamycin, azithromycin, thiabendazole, proziquantel, diethylcarbamazine, triclabendzole, quinacrine, furazolidone and rifampin, benflumetal, tiamulin, 1,2,5-oxadiazoles, 1,2 or 1,5-isoxazoles, Revlimid, and Actimid.

4. Amine acid salts of claim 3 consisting of hydrochlorides, acid phosphates, acid, sulfates, acid phosphonates, or bisphosphonates and acid phosphinates.

5. Bioactive mycobacterial anions of claim 1 consisting of triclosan, o-phenyl phenol, thymol, 2-mercapto pyridine n-oxide, dialkyldithiocarbamate, lauryl sulfonates, arachidonic acid, docosahexaenoic acid, docosanoic acid, di-2-ethylhexyl phosphoric acid, salicylate, bisphosphonate.

6. The reaction products of the free acid and free base of the cations in claim 3 and anions in claim 5 via a acid-base reaction.

7. The reaction product as claimed in claim 6, whereby the free base is Revlimid or Actimid, and the free acid is a bisphosphonate like alendronate, clodronate, etidronate, ibandronate, incadronate, minodronate, meridonate, olpadronate, pamidronate, risedronate, trfudronate, ro zoledronate in a molar ratio of from about 1:1 to about 4: respectively.
